(19) United States
(12) Patent Application Publication
(10) Pub. No.: US 2023/0406839 A1
(43) Pub. Date: Dec. 21, 2023

(54) Bifunctional Compounds And Methods Of Using The Same

(71) Applicant: The General Hospital Corporation, Boston, MA (US)

(72) Inventors: James T. Kurnick, Quincy, MA (US); Ian Seymour Dunn, Beverly, MA (US); Matthew M. Lawler, Madison, WI (US)

(21) Appl. No.: 18/035,904

(22) PCT Filed: Nov. 5, 2021
(86) PCT No.: PCT/US2021/058299

§ 371 Date: May 8, 2023

Related U.S. Application Data
(60) Provisional application No. 63/111,269, filed on Nov. 9, 2020.

(51) Int. Cl. C07D 401/12; C07D 211/32

(52) U.S. Cl. CPC C07D 401/12; C07D 211/32

(57) ABSTRACT

The present disclosure provides bifunctional compounds and methods of labeling a cell having an azide-modified sugar on its surface by contacting the cell with a bifunctional compound.

### CROSS-REFERENCE TO RELATED APPLICATIONS

This application claims priority to U.S. Provisional Patent Application No. 63/111,269, filed Nov. 9, 2020. The contents of this application are incorporated herein by reference in its entirety.

### FIELD OF THE INVENTION

The present disclosure is directed, in part, to bifunctional compounds, and methods of labeling a cell having an azide-modified sugar on its surface by contacting the cell with a bifunctional compound.

### BACKGROUND

Numerous techniques exist for assembly of a molecule on the surface of a cell or within a cell. For example, several processes for the templated assembly of molecules by proximity-enhanced reactivity have been described (see, PCI Publications WO 14/197547, WO 17/205277, WO 18/94070, WO 18/94195, WO 18/93978, and WO 19/032942). In many of these processes, a template polynucleotide is delivered to the cell, such as to the surface of the cell, whereby the template polynucleotide can serve as a substrate by which to target the cell for the assembly of a molecule. The assembled molecule can then serve, for example, as a means to destroy the cell (such as by being a toxin) or by acting as a target for a therapeutic compound (such as by being an antigen for a therapeutic antibody). Numerous methods for the delivery of the template polynucleotide are set forth in the foregoing processes. New methods for delivering a template polynucleotide to a cell and bifunctional compounds for carrying out the same are needed.

### SUMMARY

The present disclosure provides compounds having the formula:

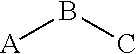

wherein: A is a small molecule ligand that binds to an FKBP binding site; B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a. maleimidyl, a hydrazone, a urethane, an azole, an imine, a haloalkyl, or a carbamate, or any combination thereof; and C is an azide reactive molecule chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane.

The present disclosure also provides methods of labeling a cell having an azide-modified sugar on its surface, the method comprising contacting the cell with a compound having the formula:

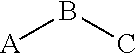

wherein: A is a small molecule ligand that binds to an FKBP binding site; B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof; and C is an azide reactive molecule chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane:

### BRIEF DESCRIPTION OF THE DRAWINGS

FIG. 1 shows azide metabolic labeling of HeLa cells, demonstrated by subsequent reaction with DBCO-FAM.

FIG. 2 shows flow detection of HeLa cells metabolically labeled with AzNAM and then reacted with DBCO-FAM, in comparison to fluorometric measurement (fluorescent plate reader) for the same cells.

FIG. 3 shows representative surface placement of a bifunctional compound on a cell having an azide-modified sugar on its surface.

FIG. 4 shows representative surface placement of an FKBP-binding compound-polynucleotide complex on a cell having a bifunctional compound bound thereto.

### DETAILED DESCRIPTION

As used herein, the term “alkyl” means a saturated hydrocarbon group which is straight-chained or branched. In some embodiments, the alkyl group has from 1 to 20 carbon atoms, from 2 to 20 carbon atoms, from 2 to 16 carbon atoms, from 4 to 12 carbon atoms, from 4 to 16 carbon atoms, from 4 to 10 carbon atoms, from 1 to 10 carbon atoms, from 2 to carbon atoms, from 1 to 8 carbon atoms, from 2 to 8 carbon atoms, from 1 to 6 carbon atoms, from 2 to 6 carbon atoms, from 1 to 4 carbon atoms, from 2 to 4 carbon atoms, from 1 to 3 carbon atoms, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, octyl, nonyl, 4,4-dimethylpentyl, 2,2,4-trimethylpentyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3 -dimethyl-l-butyl, 2-ethyl-1-butyl, and the like.

As used herein, the term “ethylene glycol unit” means a polymer of —(O—CH₂—CH₂)ₙ—O—, wherein n is from 1 to about 20. A polyethylene glycol (PEG) having 4 ethylene glycol units (i.e., —(O—CH₂—CH₂)₄—O—) is referred to herein as PEG4.

As used herein, the terms “comprising” (and any form of comprising, such as “comprise”, “comprises”, and “comprised”) and “having” (and any form of having, such as “have” and “has”) are inclusive and open-ended and include the options following the terms, and do not exclude additional, unrecited elements, or method steps.

As used herein, the term “contacting” means bringing together a compound and a cell, or a compound with another compound in an in vitro system or an in vivo system.

At various places herein, substituents of compounds may be disclosed in groups or in ranges. Designation of a range of values includes all integers within or defining the range (including the two endpoint values), and all subranges defined by integers within the range. It is specifically intended that the disclosure include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The structures depicted herein may omit necessary hydrogen atoms to complete the appropriate valency. Thus, in some instances a carbon atom or nitrogen atom may appear to have an open valency (i.e., a carbon atom with only two bonds showing would implicitly also be bonded to two hydrogen atoms; in addition, a nitrogen atom with a single bond depicted would implicitly also be bonded to two hydrogen atoms). For example, “—N” would be considered by one skilled in the art to be “—NH₂.” Thus, in any structure depicted herein wherein a valency is open, one or more hydrogen atoms, as appropriate, is implicit, and is only omitted for brevity.

The compounds described herein can also include all isotopes of atoms occurring in the intermediates or final compounds. Isotopes include those atoms having the same atomic number but different mass numbers. For example, isotopes of hydrogen include tritium and deuterium. Carbon (¹²C) can be replaced at any position with ¹³C or ¹⁴C. Nitrogen (¹⁴N) can be replaced with ¹⁵N. Oxygen (¹⁶O) can be replaced at any position with ¹⁷O or ¹⁸O. Sulfur (³²S) can be replaced with ³³S, ³⁴S or ³⁶S. Chlorine (³⁵Cl) can be replaced with ³⁷Cl. Bromine (⁷⁹Br) can be replaced with ⁸¹Br.

In some embodiments, the compounds, or salts thereof, are substantially isolated. Partial separation can include, for example, a composition enriched in any one or more of the compounds described herein. Substantial separation can include compositions containing at least about 50%, at least about 60%, at least about 70%, at least about 80%, at least about 90%, at least about 95%, at least about 97%, or at least about 99% by weight of any one or more of the compounds described herein, or salt thereof. Methods for isolating compounds and their salts are routine in the art.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The present disclosure provides bifunctional compounds having the formula:

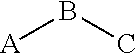

wherein:

A is a small molecule ligand that binds to an FKBP binding site;

B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof; and

C is an azide reactive molecule chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane.

In some embodiments, the FKBP binding site to which the small molecule ligand A binds is the FK506-FKBP binding site or the mutant (F36V) FKBP binding site. In some embodiments, the FKBP binding site is the FK506-FKBP binding site. In some embodiments, the FKBP binding site is the mutant (F36V) FKBP binding site. In some embodiments, the small molecule ligand (A) is

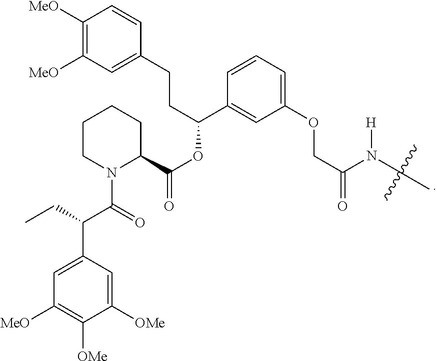 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

In some embodiments, the chemical linker B is chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof. In some embodiments, the chemical linker is chosen from an alkyl, an alkenyl, an amide, an ester, a thioester, a ketone, an ether, a thioether, a disulfide, an ethylene glycol unit, a haloalkyl, or a carbamate. In some embodiments, the chemical linker is chosen from an alkyl, an alkenyl, an amide, an ester, a thioester, a disulfide, an ethylene glycol unit, or a haloalkyl. In some embodiments, the chemical linker is chosen from an alkyl, an alkenyl, an amide, an ethylene glycol unit, or a haloalkyl. In some embodiments, the chemical linker is an alkyl or an ethylene glycol unit. In some embodiments, the chemical linker is an alkyl. In some embodiments, the alkyl is a C₂-C₁₆alkyl. In some embodiments, the alkyl is a C₄-C₁₂alkyl or a C₄-C₁₆alkyl. In some embodiments, the alkyl is a C₄-C₁₀alkyl, in some embodiments, the alkyl is C₄alkyl or C₁₀alkyl. In some embodiments, the chemical linker is an ethylene glycol unit. In some embodiments, the ethylene glycol unit is a polyethylene glycol (PEG). In some embodiments, the ethylene glycol unit is PEG2 to PEG16. In some embodiments, the ethylene glycol unit is PEG2, PEG3, or PEG4.

In some embodiments, the azide reactive molecule C is chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane. In some embodiments, the azide reactive molecule is chosen from a cyclooctyne, a norbornene, a phosphine, a diallkyl phosphine, a trialkyl phosphine, a cyclooctene, a tetrazine, a tetrazole, or a quadricyclane. In some embodiments, the azide reactive molecule is chosen from a cyclooctyne, a norbornene, a phosphine, a cyclooctene, a tetrazine, or a tetrazole. In some embodiments, the azide reactive molecule is chosen from a cyclooctyne, a cyclooctene, and a tetrazine. In some embodiments, the cyclooctyne is dibenzocyclooctyne (DBCO), bicyclo[6.1.0]nonyne (BCN), monofluorinated cyclooctyne, difluorocyclooctyne, dimethoxyazacyclooctyne, dibenzoazacyclooctyne, biarytazacyclooctynone, 2,3,6,7-tetramethoxy-dibenzocyclooctyne, sulfonylated dibenzocyclooctyne, carboxymethylmonobenzocyclooctyne, or pyrrolocyclooctyne. In some embodiments, the cyclooctyne is DBCO or BCN. In some embodiments, the cyclooctene is trans-cyclooctene (TCO). In some embodiments, the tetrazine is methyltetrazine, diphenyltetrazine, 3,6-di-(2-pyridyl)-s-tetrazine, 3,6-diphenyl-s-tetrazine, 3-(5-aminopyridin-2-yl)-6-(pyridin-2-yl)-s-tetrazine, or N-benzoyl-3-(5-aminopyridin-2-yl)-6-(pyridin-2-yl)-s-tetrazine. In some embodiments, the tetrazine is methyltetrazine or diphenyttetrazine. In some embodiments, the tetrazine is methyltetrazine.

In some embodiments, the azide reactive molecule (portion C of the bifunctional compounds described herein) can be replaced with an alkyne reactive molecule, a halogen reactive molecule, a short-chain alkyl group reactive molecule, a halogenated alkyl group reactive molecule, a sulfhydryl reactive molecule, a thiomethyl group reactive molecule, a cyclopropene reactive molecule, or a cyclopentene reactive molecule. Portion C of the bifunctional molecule in these embodiments would be able to chemically react with alkynes, halogens, short-chain alkyl groups, halogenated alkyl groups, sulfhydryls, thiomethyl groups, cyclopropenes, and cyclopentenes, respectively.

In some embodiments of the bifunctional compound, the FKBP binding site is the FK506-FKBP binding site or the mutant (F36V) FKBP binding site; the chemical linker is an alkyl or an ethylene glycol unit; and the azide reactive molecule is chosen from a cyclooctyne, a cyclooctene, and a tetrazine.

In some embodiments of the bifunctional compound, the FKBP binding site is the FK506-FKBP binding site or the mutant (F36V) FKBP binding site; the chemical linker is a C₂-C₁₆alkyl or a polyethylene glycol which is PEG2 to PEG16; and the azide reactive molecule is DBCO, BCN, TCO, or methyltetrazine.

In some embodiments of the bifunctional compound, the FKBP binding site is the mutant (F36V) FKBP binding site; the chemical linker is a C₄-C₁₀alkyl or a polyethylene glycol which is PEG2, PEG3, or PEG4; and the azide reactive molecule is DBCO, BCN, TCO or methyltetrazine.

In some embodiments of the bifunctional compound, the small molecule ligand (A) is

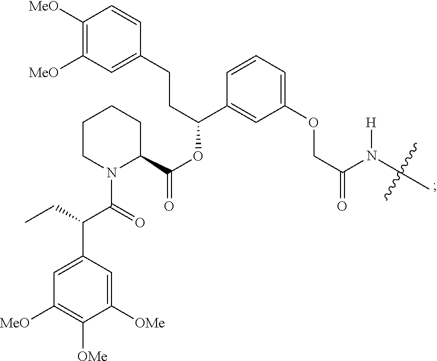 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

the chemical linker is C₄alkyl, C₁₀alkyl, or PEG3; and the azide reactive molecule is DBCO or BCN.

In some embodiments of the bifunctional compound, the compound comprises the formula:

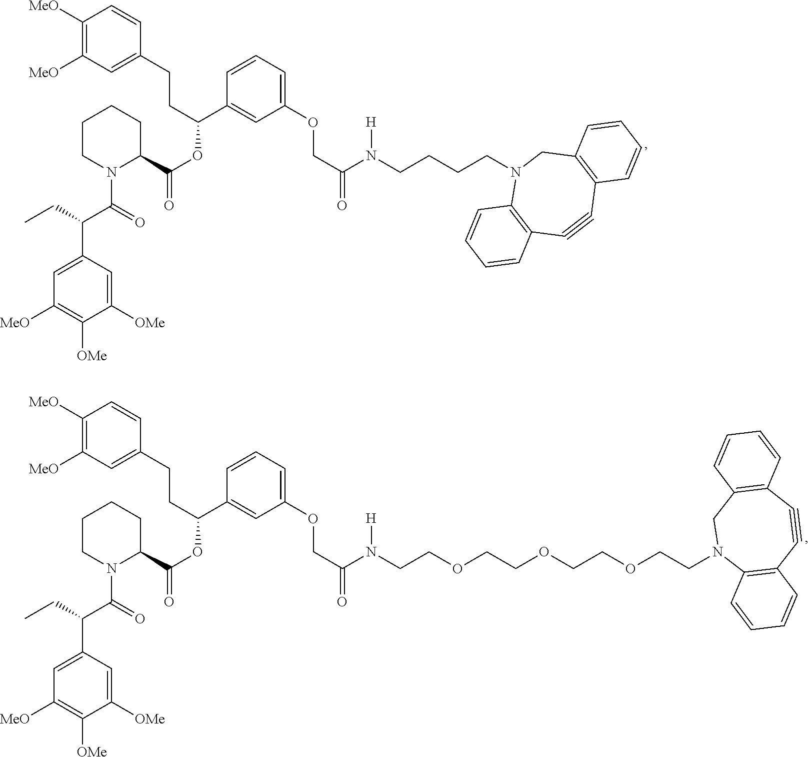 [SMILES: CC[C@H](C(=O)N1CCCC[C@H]1C(=O)O[C@H](CCc1ccc(OC)c(OC)c1)c1cccc(OCC(=O)NCCCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCN2Cc3ccccc3C#Cc3ccccc32)c1)c1cc(OC)c(OC)c(OC)c1]

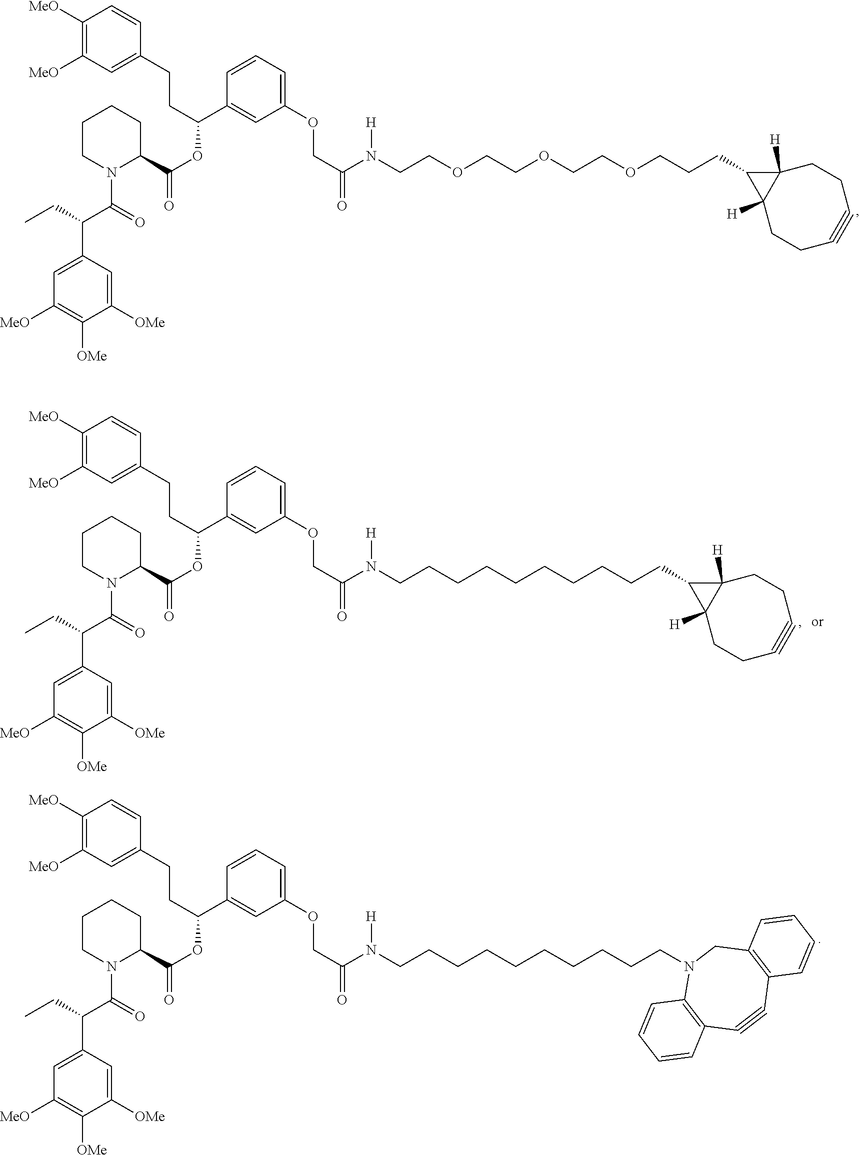

The present disclosure also provides methods of labeling a cell having an azide-modified sugar on its surface. The methods comprise contacting the cell with a bifunctional compound having the formula:

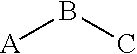

wherein:

A is a small molecule ligand that binds to an FKBP binding site;

B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleirnidyl, a hydrazone, a urethane, an azole, an imine, a haloalkyl, or a carbamate, or any combination thereof; and

C is an azide reactive molecule chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane. In some embodiments, the methods further comprise contacting the cell with the azide-modified sugar prior to contacting the cell with the bifunctional compound. In some embodiments, the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA). In some embodiments, the azide-modified sugar is AzNAM. In some embodiments, the azide-modified sugar is AzGlcNAc. In some embodiments, the azide-modified sugar is AGalNAc. In some embodiments, the azide-modified sugar is AzNANA. In some embodiments, the azide-modified sugar is acetylated at 1, 2, 3, or 4 positions. In some embodiments, the azide-modified sugar is acetylated at 1 position. In some embodiments, the azide-modified sugar is acetylated at 2 positions. In some embodiments, the azide-modified sugar is acetylated at 3 positions. In some embodiments, the azide-modified sugar is acetylated at 4 positions.

In some embodiments, the methods further comprise contacting the cell having an azide-modified sugar on its surface with a complex. In some embodiments, the complex comprises an FKBP binding site linked to a polynucleotide, peptide, or small molecule. In some embodiments, the complex comprises an FKBP binding site linked to a polynucleotide. In some embodiments, the complex comprises an FKBP binding site linked to a peptide. In some embodiments, the complex comprises an FKBP binding site linked to a small molecule. In some embodiments, the FKBP binding site is the FK506-FKBP binding site or the mutant (F36V) FKBP binding site. In some embodiments, the FKBP binding site is the FK506-FKBP binding site. In some embodiments, the FKBP binding site is the mutant (F36V) FKBP binding site. In some embodiments, the complex comprises the FK506-FKBP binding site linked to a polynucleotide. In some embodiments, the complex comprises the mutant (F36V) FKBP binding site linked to a polynucleotide. The polynucleotide portion of the complex can serve as, for example, a template polynucleotide for a template assembly by proximity-enhanced reactivity process to occur.

In any of the methods described herein, the FKBP binding site to which the small molecule ligand A binds is the FK506-FKBP binding site or the mutant (F36V) FKBP binding site. In some embodiments, the FKBP binding site is the FK506-FKBP binding site. In some embodiments, the FKBP binding site is the mutant (F36V) FKBP binding site. In some embodiments, the small molecule ligand is

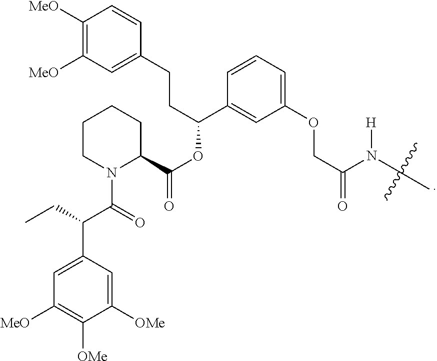 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

In any of the methods described herein, the chemical linker B is chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof. In some embodiments, the chemical linker is chosen from an alkyl, an alkenyl, an amide, an ester, a thioester, a ketone, an ether, a thioether, a disulfide, an ethylene glycol unit, a haloalkyl, or a carbamate. In some embodiments, the chemical linker is chosen from an alkyl, an alkenyl, an amide, an ester, a thioester, a disulfide, an ethylene glycol unit, or a haloalkyl. In some embodiments, the chemical linker is chosen from an alkyl, an alkenyl, an amide, an ethylene glycol unit, or a haloalkyl. In some embodiments, the chemical linker is an alkyl or an ethylene glycol unit. In some embodiments, the chemical linker is an alkyl. In some embodiments, the alkyl is a C₂-C₁₆alkyl. In some embodiments, the alkyl is a C₄-C₁₂alkyl or a C₄-C₁₆alkyl. In some embodiments, the alkyl is a C₄-C₁₀alkyl. In some embodiments, the alkyl is C₄alkyl or C₁₀alkyl. In some embodiments, the chemical linker is an ethylene glycol unit. In some embodiments, the ethylene glycol unit is a polyethylene glycol (PEG). In some embodiments, the ethylene glycol unit is PEG2 to PEG16. In some embodiments, the ethylene glycol unit is PEG2, PEG3, or PEG4.

In any of the methods described herein, the azide reactive molecule C is chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane. In some embodiments, the azide reactive molecule is chosen from a cyclooctyne, a norbornene, a phosphine, a dialkyl phosphine, a trialkyl phosphine, a cyclooctene, a tetrazine, a tetrazole, or a quadricyclane. In some embodiments, the azide reactive molecule is chosen from a cyclooctyne, a norbornene, a phosphine, a cyclooctene, a tetrazine, or a tetrazole. In some embodiments, the azide reactive molecule is chosen from a cyclooctyne, a cyclooctene, and a tetrazine. In some embodiments, the cyclooctyne is dibenzocyclooctyne (DBCO), bicyclo[6.1.0]nonyne (BCN), monofluorinated cyclooctyne, difluorocyclooctyne, dimethoxyazacyclooctyne, dibenzoazacyclooctyne, biarylazacyclooctynone, 2,3,6,7-tetramethoxy-dibenzocyclooctyne, sulfonylated dibenzocyclooctyne, carboxymethylmonobenzocyclooctyne, or pyrrolocyclooctyne. In some embodiments, the cyclooctyne is DBCO or BCN. In some embodiments, the cyclooctyne is DBCO. In some embodiments, the cyclooctyne is BCN. In some embodiments, the cyclooctene is trans-cyclooctene (TCO). In some embodiments, the tetrazine is methyltetrazine, diphenyltetrazine, 3,6-di-(2-pyridyl)-s-tetrazine, 3,6-diphenyl-s-tetrazine, 3-(5-aminopyridin-2-yl)-6-(pyridin-2-yl)-s-tetrazine, or N-benzoyl-3-(5-aminopyridin-2-yl)-6-(pyridin-2-yl)-s-tetrazine. In some embodiments, the tetrazine is methyltetrazine or diphenyltetrazine. In some embodiments, the tetrazine is methyltetrazine, In any of the methods described herein, the azide reactive molecule (portion C of the bifunctional compounds described herein) can be replaced with an alkyne reactive molecule, a halogen reactive molecule, a short-chain alkyl group reactive molecule, a halogenated alkyl group reactive molecule, a sulfhydryl reactive molecule, a thiomethyl group reactive molecule, a cyclopropene reactive molecule, or a cyclopentene reactive molecule. Portion C of the bifunctional molecule in these embodiments would be able to chemically react with alkynes, halogens, short-chain alkyl groups, halogenated alkyl groups, sulfhydryls, thiomethyl groups, cyclopropenes, and cyclopentenes, respectively. In such methods, instead of having an azide-modified sugar on its surface, the cell would have an alkyne-modified sugar, a halogen-modified sugar, a short-chain alkyl-modified sugar, a halogenated alkyl-modified sugar, a sulfhydryl-modified sugar, a thiomethyl-modified sugar, a cyclopropene-modified sugar, or a cyclopentene-modified sugar, respectively, on its surface (or be contacted by such a modified sugar).

In any of the methods described herein, for the bifunctional compound, the FKBP binding site is the FK506-FKBP binding site or the mutant (F36V) FKBP binding site; the chemical linker is an alkyl or an ethylene glycol unit; and the azide reactive molecule is chosen from a cyclooctyne, a cyclooctene, and a tetrazine.

In any of the methods described herein, for the bifunctional compound, the FKBP binding site is the FK506-FKBP binding site or the mutant (F36V) FKBP binding site; the chemical linker is a C₂-C₁₆alkyl or a polyethylene glycol which is PEG2 to PEG16; and the azide reactive molecule is DBCO, BCN, TCO, or methyltetrazine.

In any of the methods described herein, for the bifunctional compound, the FKBP binding site is the mutant (F36V) FKBP binding site; the chemical linker is a C₄-C₁₀alkyl or a polyethylene glycol which is PEG2, PEG3, or PEG4; and the azide reactive molecule is DBCO, BCN, TCO, or methyltetrazine.

In any of the methods described herein, for the bifunctional compound, the small molecule ligand is

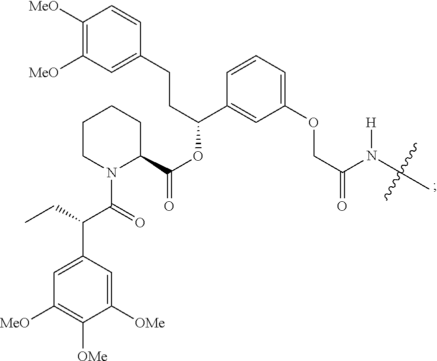 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

the chemical linker is C₄alkyl, C₁₀alkyl, or PEG3; and the azide reactive molecule is DBCO or BCN.

In any of the methods described herein, the bifunctional compound comprises the formula:

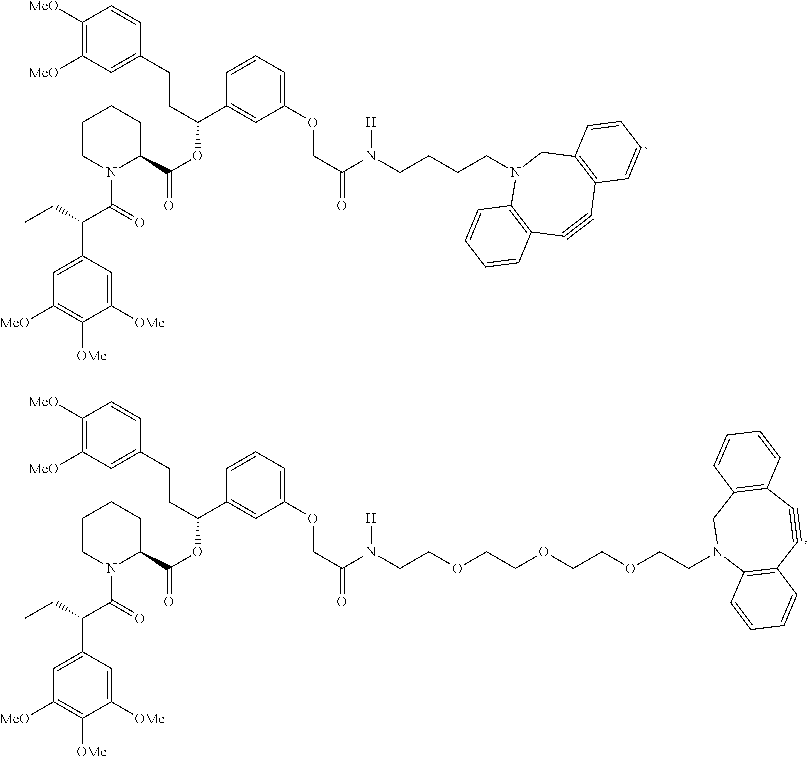 [SMILES: CC[C@H](C(=O)N1CCCC[C@H]1C(=O)O[C@H](CCc1ccc(OC)c(OC)c1)c1cccc(OCC(=O)NCCCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCN2Cc3ccccc3C#Cc3ccccc32)c1)c1cc(OC)c(OC)c(OC)c1]

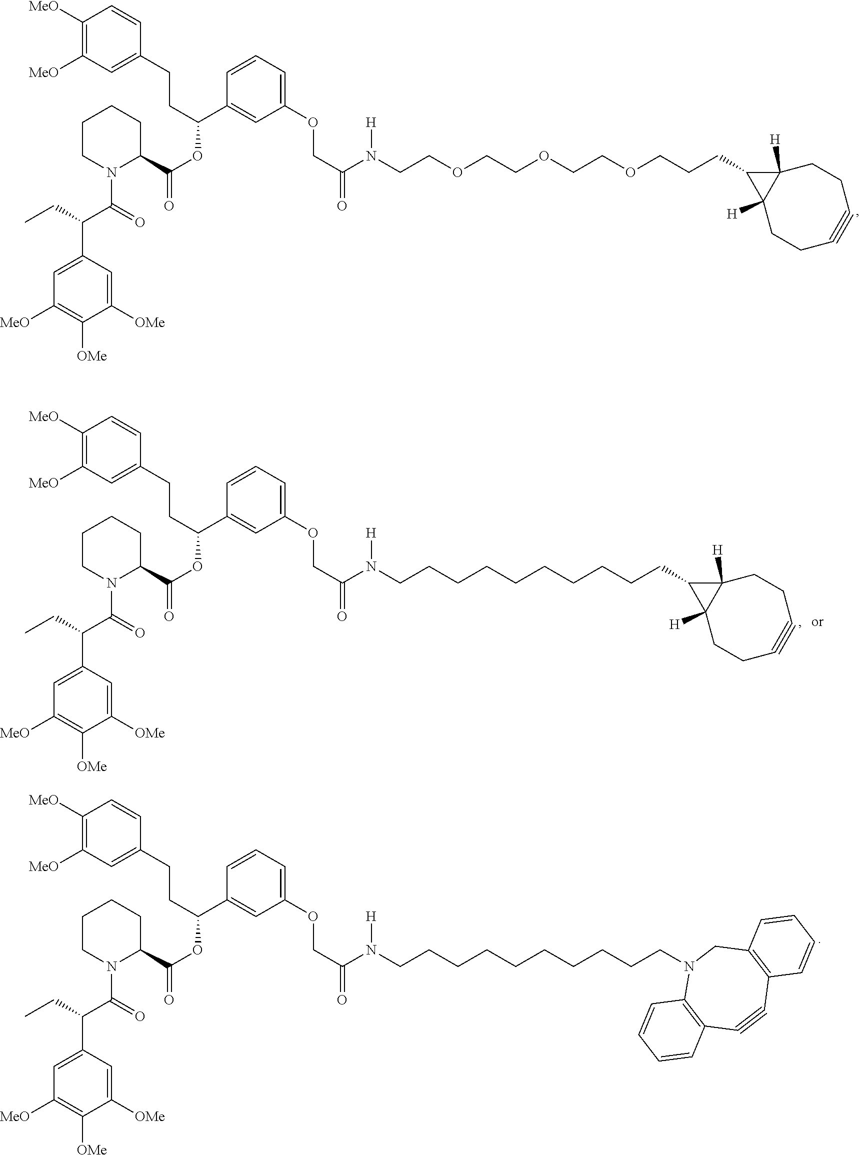

In some embodiments, the complex comprising the FKBP binding site linked to a polynucleotide, peptide, or small molecule can be pre-incubated with a bifunctional compound (such as an excess amount of bifunctional compound) prior to exposure to the target cells displaying surface azide. The resulting complex-bifunctional compound can then used to treat cells having surface azide.

In any of the methods described herein, the cell can be any desired target cell. In some embodiments, the cell is a virus infected cell, a tumor cell, a cell infected with a microbe, or a cell that produces a molecule that leads to a disease, such as a cell that produces an antibody that induces allergy, anaphylaxis, or autoimmune disease, or a cytokine that mediates a disease. The cells described herein can be contacted with any of the azide-modified sugars described herein either in vitro or in vivo. The cells described herein can also be contacted with any of the bifunctional compounds described herein either in vitro or in vivo. The cells described herein can also be contacted with any of the complexes described herein either in vitro or in vivo.

When any of the compounds described herein, including the azide-modified sugars, the bifunctional compounds, and/or the complexes, are contacted with a cell in vivo, the compounds can be delivered to a mammal, such as a human, by numerous routes of administration. Suitable routes of administration include, but are not limited to, oral, sublingual, buccal, rectal, intranasal, inhalation, eye drops, ear drops, epidural, intracerebral, intracerebroventricular, intrathecal, epicutaneous, transdermal, subcutaneous, intradermal, intravenous, intraarterial, intraosseous infusion, intramuscular, intracardiac, intraperitoneal, intravesical infusion, and intravitreal. In some embodiments, the administration is oral, sublingual, buccal, rectal, intranasal, inhalation, eye drops, or ear drops. In some embodiments, the administration is oral, sublingual, buccal, rectal, intranasal, or inhalation. In some embodiments, the administration is epidural, intracerebral, intracerebroventricular, or intrathecal. In some embodiments, the administration is epicutaneous, transdermal, subcutaneous, or intradermal. In some embodiments, the administration is intravenous, intraarterial, intraosseous infusion, intramuscular, intracardiac, intraperitoneal, intravesical infusion, or intravitreal. In some embodiments, the administration is intravenous, intramuscular, or intraperitoneal. The route of administration can depend on the particular disease, disorder, or condition being treated and can be selected or adjusted by the clinician according to methods known to the clinician to obtain desired clinical responses. Methods for administration are known in the art and one skilled in the art can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In some embodiments, it may be desirable to administer one or more compounds, or a pharmaceutically acceptable salt thereof, to a particular area in need of treatment. This may be achieved, for example, by local infusion (for example, during surgery), topical application (for example, with a wound dressing after surgery), by injection (for example, by depot injection), catheterization, by suppository, or by an implant (for example, where the implant is of a porous, non-porous, or gelatinous material, including membranes, such as sialastic membranes, or fibers). Formulations for injection can be presented in unit dosage form, such as in ampoules or in multi-dose containers, with an added preservative.

The compounds described herein can be formulated for parenteral administration by injection, such as by bolus injection or continuous infusion. The compounds can be administered by continuous infusion subcutaneously over a period of about 15 minutes to about 24 hours. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations including, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, including, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

In transdermal administration, the compounds can be applied to a plaster, or can be applied by transdermal, therapeutic systems that are consequently supplied to the organism. In some embodiments, the compounds are present in creams, solutions, powders, fluid emulsions, fluid suspensions, semi-solids, ointments, pastes, gels, jellies, and foams, or in patches containing any of the same.

The compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compounds described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

The amount of compound to be administered may be that amount which is effective to produce sufficient cell labeling. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

Suitable dosage ranges for oral administration include, but are not limited to, from about 0.001 mg to about 200 mg, from about 0.01 mg to about 100 mg, from about 0.01 mg to about 70 mg, from about 0.1 mg to about 50 mg, from 0.5 mg to about 20 mg, or from about 1 mg to about 10 mg. In some embodiments, the oral dose is about 5 mg.

Suitable dosage ranges for intravenous administration include, but are not limited to, from about 0.01 mg to about 500 mg, from about 0.1 mg to about 100 mg, from about 1 mg to about 50 mg, or from about 10 mg to about 35 mg.

Suitable dosage ranges for other routes of administration can be calculated based on the forgoing dosages as known by one skilled in the art. For example, recommended dosages for intradermal, intramuscular, intraperitoneal, subcutaneous, epidural, sublingual, intracerebral, transdermal, or inhalation are in the range from about 0.001 mg to about 200 mg, from about 0.01 mg to about 100 mg, from about 0.1 mg to about 50 mg, or from about 1 mg to about 20 mg. Effective doses may be extrapolated from dose-response curves derived from in vitro or animal model test systems.

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, the composition is in the form of a liquid wherein the active agent (i.e., one of the facially amphiphilic polymers or oligomers disclosed herein) is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is an in situ gellable aqueous solution, suspension or solution/suspension, comprising about from 0.2% to about 3% or from about 0.5% to about 1% by weight of a gelling polysaccharide, chosen from gellan gum, alginate gum and chitosan, and about 1% to about 50% of a water-soluble film-forming polymer, preferably selected from alkylcelluloses (e.g., methylcellulose, ethylcellulose), hydroxyalkylcelluloses (e.g., hydroxyethylcellulose, hydroxypropyl methylcellulose), hyaluronic acid and salts thereof, chondroitin sulfate and salts thereof, polymers of acrylamide, acrylic acid and polycyanoacrylates, polymers of methyl methacrylate and 2-hydroxyethyl methacrylate, polydextrose, cyclodextrins, polydextrin, maltodextrin, dextran, polydextrose, gelatin, collagen, natural gums (e.g., xanthan, locust bean, acacia, tragacanth and carrageenan gums and agar), polygalacturonic acid derivatives (e.g., pectin), polyvinyl alcohol, polyvinylpyrrolidone and polyethylene glycol. The composition can optionally contain a gel-promoting counterion such as calcium in latent form, for example encapsulated in gelatin.

Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the compositions. Suitable antioxidants include, but are not limited to, ascorbic acid, sodium metabisulfite, sodium bisulfite, acetylcysteine, polyquaternium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

Suitable solubilizing agents for solution and solution/suspension compositions are cyclodextrins. Suitable cyclodextrins can be chosen from α-cyclodextrin, β-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxy-alkylcyclodextrins (e.g., carboxymethyl-β-cyclodextrin), sulfoalkylether cyclodextrins (e.g., sulfobutylether-β-cyclodextrin), and the like. Applications of cyclodextrins have been reviewed in Rajewski et al., J. Pharm. Sci., 1996, 85, 1155-1159.

In some embodiments, the composition optionally contains a suspending agent. For example, in those embodiments in which the composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers. However, in some embodiments, the compositions do not contain substantial amounts of solid particulate matter, whether of the anti-microbial polymer or oligomer active agent, an excipient, or both.

One or more acceptable pH adjusting agents and/or buffering agents can be included in the compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

One or more acceptable salts can be included in the compositions in an amount required to bring osmolality of the composition into an acceptable range. Such salts include, but are not limited to, those having sodium, potassium or ammonium cations and chloride, citrate, ascorbate, borate, phosphate, bicarbonate, sulfate, thiosulfate or bisulfite anions. In some embodiments, salts include sodium chloride, potassium chloride, sodium thiosulfate, sodium bisulfite and ammonium sulfate. In some embodiments, the salt is sodium chloride.

Optionally one or more acceptable surfactants, preferably nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Labeling Cells With Azide-Modified Sugars

Demonstration of the presence of azides on a cell surface can be achieved by treating a cell preparation with a fluorescent bio-orthogonal reagent that reacts only with the azide group, without chemical reactivity with normal biological molecules. Initially, cells are cultured in a suitable culture vessel such that their level of confluency at the time of addition of the azide-modified sugar AzNAM is not more than 80%.

In particular, HeLa cells were plated in 6-well plates (2.5×10⁴ cells/well) and incubated for 48 hours in DMEM-10% FBS medium in a standard 5% CO₂ atmosphere. Medium from each well was removed, and each well was washed with 2 ml of phosphate buffered saline (PBS), and fresh DMEM-FBS (1 ml) added. Subsequently, varying amounts of AzNAM (solubilized in DMSO) were added to the wells in small (10 μl) volumes to produce the desired final concentrations in the range 25-125 μM. After an additional 20 hours incubation, the medium in each well was removed, and the wells were washed with 2 ml of PBS, followed by addition of 1 ml of PBS. Then, a fluorescent bio-orthogonally azide-reactive reagent DBCO-FAM (Broadpharm) was directly added to each well to produce a final concentration of 10 μM.

Plates with added DBCO-FAM were protected from bright light and incubated for 1 hour at room temperature. Supernatants in each well were then removed, and each well was washed twice with 2 ml of PBS. Cells were then taken up with CellStripper (Thermo) reagent and transferred into 1.5 ml tubes. After pelleting and washing in PBS, cells were resuspended in 150 μl of PBS and counted. Defined numbers in 50 μl volumes in a 96-well Blackwell plate (Corning) were assayed for fluorescence in a fluorescent plate reader (Tecan).

Fluorescence arising from the reaction between DBCO-FAM and cell-surface azide label was also demonstrated by flow cytometry. In separate trials, HeLa cells were added to wells of a 12-well plate at 10,000 cells/well, and cultured for 72 hours under normal conditions. The wells were then washed with 1 ml of PBS, and 0.5 ml of DMEM-10% FBS was added. AzNAM was then added to a final concentration of 125 μM, with cells receiving no azide-modified sugar as controls. After 20 hours, cells were harvested with CellStripper (250 μl/well), washed with PBS, and subjected to flow analysis.

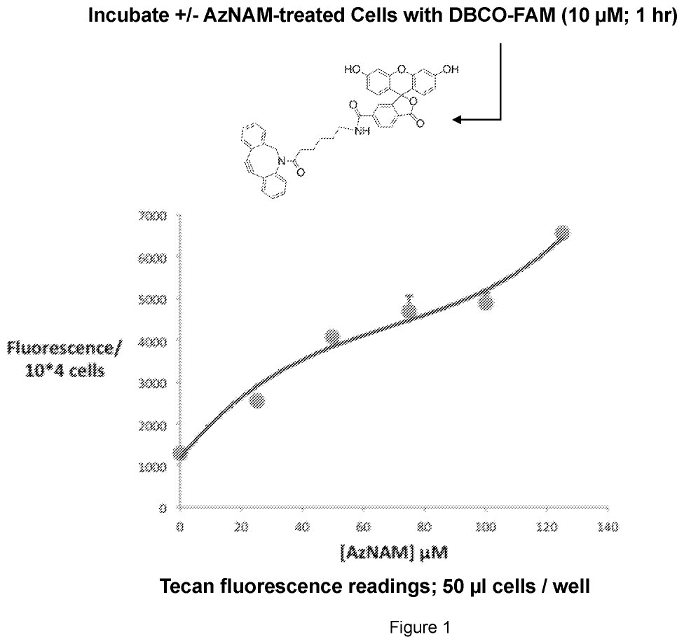

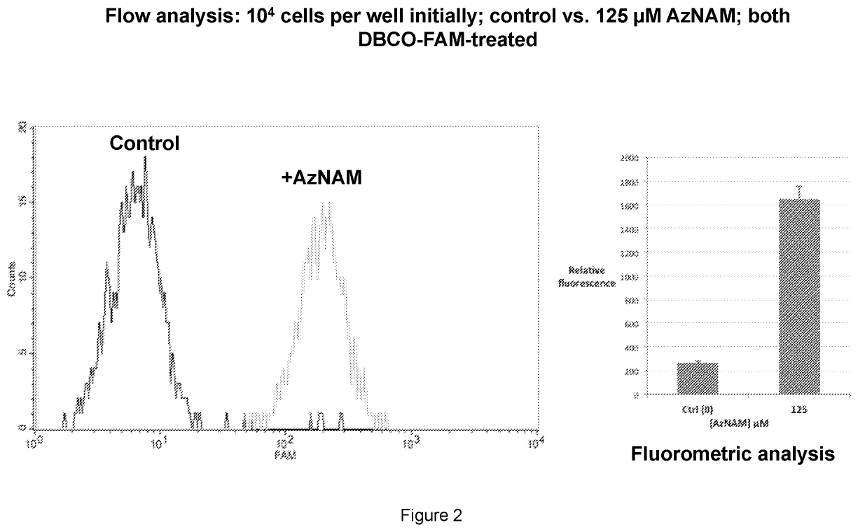

By fluorescence readings in a 96-well plate format, it was demonstrated that cell-associated fluorescence increased as a function of the levels of AzNAM used in the initial labeling test (see, FIG. 1). With flow analyses, marked and well-demarcated peak shifts were observed with the AzNAM-treated cells, which was corroborated by measuring fluorescence from equal numbers of cells in a Tecan fluorescent plate reader (see, FIG. 2).

### Example 2

### Labeling Cells Having Surface Azide-Modified Sugars with Bifunctional Compounds

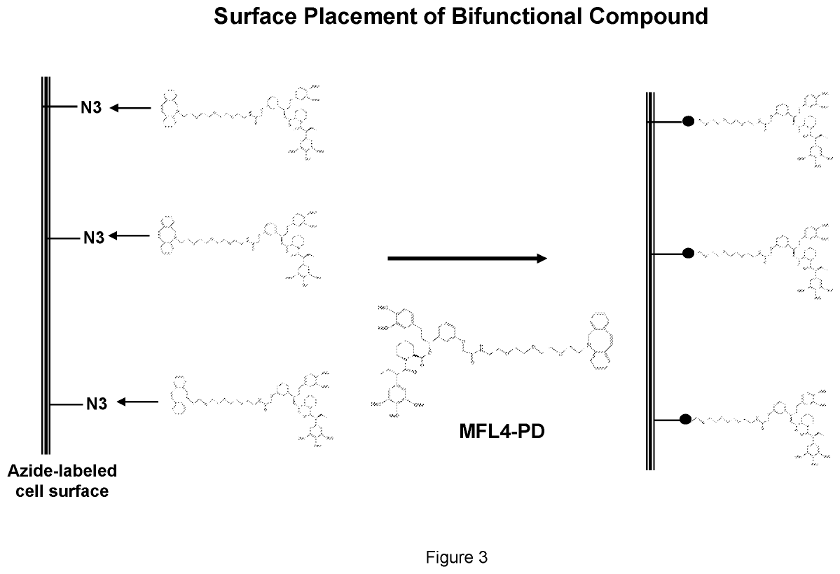

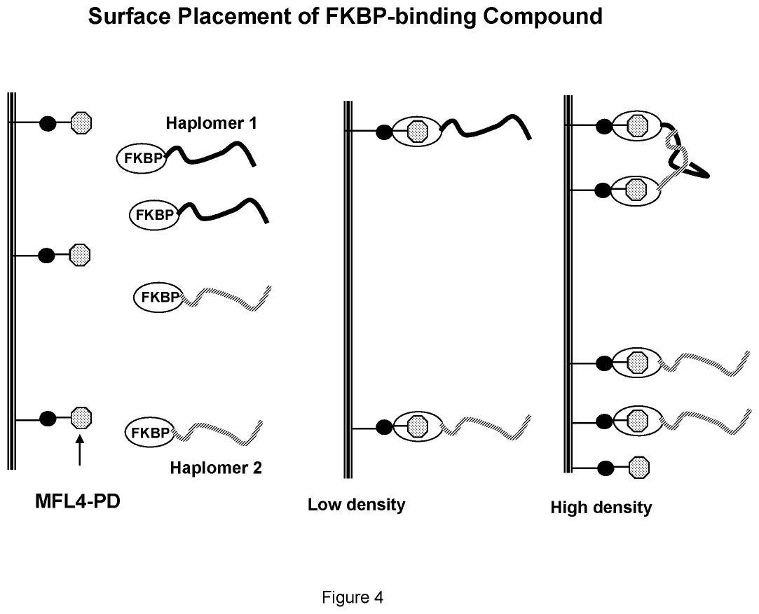

Cells that have been metabolically labeled with surface azide-modified sugars (see, Example 1) can be subsequently reacted with the bifunctional compounds described herein (see, FIG. 3). After such reactions have occurred and excess compound is removed, the portion of the bifunctional molecule that is a small molecule ligand that binds to an FKBP binding site (portion A) may be displayed on the surface of the cell and may be available for subsequent reactions with a complex comprising an FKBP binding site linked to a polynucleotide, peptide, or small molecule (see, FIG. 4). Alternately, the portion of the bifunctional molecule that is a small molecule ligand that binds to an FKBP binding site (portion A) may first be allowed to react with the complex comprising an FKBP binding site linked to a polynucleotide, peptide, or small molecule, after which the exposed azide reactive molecule (portion C) of the bifunctional compound is used for targeting the complex to the cell surface labeled with azide-modified sugars.

In particular, any of the bifunctional compounds described herein can be used for the purposes of cell surface positioning of any of the complexes described herein. In this Example, cells displaying azide moieties on surface glycan molecules (as in Example 1) are treated with 1 mM of the bifunctional compound (initially solubilized in DMSO as a 100 mM stock solution and diluted accordingly to the final desired concentration) in serum-free RPMI medium for 2 hours at room temperature in the presence of 1 mg/ml bovine serum albumin (BSA) (Sigma) and 500 μg/ml salmon sperm DNA. This treatment is followed by centrifugation (5 minutes at 2000 rpm in an Eppendorf centrifuge), followed by two washes with serum-free RPMI medium, with resuspension in 100 μl of the same medium. Following this, a complex comprising an FKBP binding site linked to a polynucleotide, peptide, or small molecule is added to the bifunctional compound-modified cells at a concentration of 1 pmol/μl, for a one hour incubation at room temperature. The cell preparations are then repelleted, washed twice with serum-free RPMI medium and once with PBS, with a final resuspension in 100 μl of PBS.

In an alternate embodiment, the complex comprising an FKBP binding site linked to a polynucleotide, peptide, or small molecule is pre-incubated with excess bifunctional compound prior to exposure to the target cells displaying surface azide. The complex in PBS (100 pmol) is incubated with a 10-fold molar excess of the bifunctional compound for one hour at room temperature, followed by passage through a PBS-equilibrated P6 desalting column (Bio-Rad) to remove excess bifunctional compound. The resulting complex-bifunctional compound is then used to treat cells having surface azide, followed by washing steps as above.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A compound haying the formula: 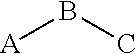
wherein:
A is a small molecule ligand that binds to an FKBP binding site;
B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof; and
C is an azide reactive molecule chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane.

2. The compound according to claim 1, wherein the FKBP binding site is the FK506-FKBP binding site or the mutant (F36V) FKBP binding site.

3. The compound according to claim 1 or claim 2, wherein the FKI P binding site is the FK506-FKBP binding site.

4. The compound according to claim 1 or claim 2, wherein the FKBP binding site is the mutant (F36V) FKBP binding site.

5. The compound according to claim 1 or claim 2, wherein the small molecule ligand is

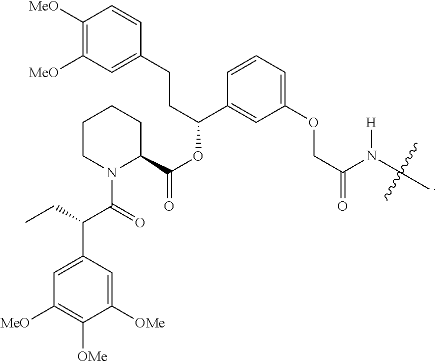 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

6. The compound according to any one of claims 1 to 5, wherein the chemical linker is an alkyl or an ethylene glycol unit.

7. The compound according to claim 6, wherein the chemical linker is an alkyl.

8. The compound according to claim 7, wherein the chemical linker is a C₂-C₁₆alkyl.

9. The compound according to claim 8, wherein the chemical linker is a C₄-C₁₂alkyl or a C₄-C₁₆alkyl.

10. The compound according to claim 9, wherein the chemical linker is a C₄-C₁₀alkyl.

11. The compound according to claim 10, wherein the chemical linker is C₄alkyl or C₁₀alkyl.

12. The compound according to claim 6, wherein the chemical linker is an ethylene glycol unit.

13. The compound according to claim 12, wherein the chemical linker is a polyethylene glycol (PEG).

14. The compound according to claim 13, wherein the PEG is PEG2 to PEG16.

15. The compound according to claim 14, wherein the PEG is PEG2, PEG3, or PEG4.

16. The compound according to any one of claims 1 to 15, wherein the azide reactive molecule is chosen from a cyclooctyne, a cyclooctene, and a tetrazine.

17. The compound according to claim 16, wherein the cyclooctyne is dibenzocyclooctyne (DBCO), bicyclo[6.1.0]nonyne (BCN), monofluorinated cyclooctyne, difluorocyclooctyne, dimethoxyazacyclooctyne, dibenzoazacyclooctyne, biarylazacyclooctynone, 2,3,6,7-tetramethoxy-dibenzocyclooctyne, sulfonylated dibenzocyclooctyne, carboxymethylmonobenzocyclooctyne, or pyrrolocyclooctyne.

18. The compound according to claim 16, wherein the cyclooctene is trans-cyclooctene (TCO).

19. The compound according to claim 16, wherein the tetrazine is methyltetrazine, diphenyltetrazine, 3,6-di-(2-pyridyl)-s-tetrazine, 3,6-diphenyl-s-tetrazine, 3-(5-aminopyridin-2-yl)-6-(pyridin-2-yl)-s-tetrazine, or N-benzoyl-3-(5-aminopyridin-2-yl)-6-(pyridin-2-yl)-s-tetrazine.

20. The compound according to claim 1, wherein:
the FKBP binding site is the FK506-FKBP binding site or the mutant (F36V) FKBP binding site;
the chemical linker is an alkyl or an ethylene glycol unit; and
the azide reactive molecule is chosen from a cyclooctyne, a cyclooctene, and a tetrazine.

21. The compound according to claim 1, wherein:
the FKBP binding site is the FK506-FKBP binding site or the mutant (F36V) FKBP binding site;
the chemical linker is a C₂-C₁₆alkyl, or a polyethylene glycol which is PEG2 to PEG16; and
the azide reactive molecule is DBCO, BCN, TCO, or methyltetrazine.

22. The compound according to claim 1, wherein:
the FKBP binding site is the mutant (F36V) FKBP binding site;
the chemical linker is a C₄-C₁₀alkyl or a polyethylene glycol which is PEG2 PEG3, or PEG4; and
the azide reactive molecule is DBCO, BCN, TCO, or methyltetrazine.

23. The compound according to claim 1, wherein:
the small molecule ligand is 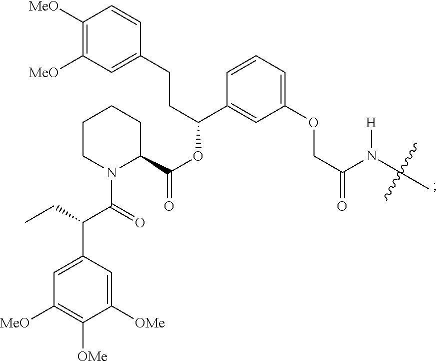 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]
the chemical linker is C₄alkyl, C₁₀alkyl, or PEG3; and
the azide reactive molecule is DBCO or BCN.

24. The compound according to claim 1, having the formula:

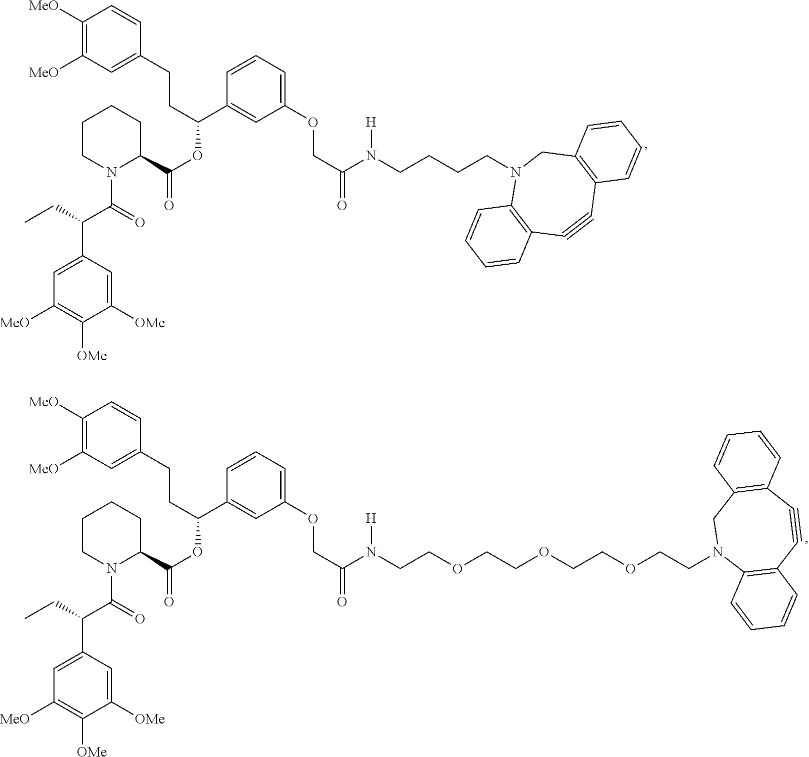 [SMILES: CC[C@H](C(=O)N1CCCC[C@H]1C(=O)O[C@H](CCc1ccc(OC)c(OC)c1)c1cccc(OCC(=O)NCCCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCN2Cc3ccccc3C#Cc3ccccc32)c1)c1cc(OC)c(OC)c(OC)c1]

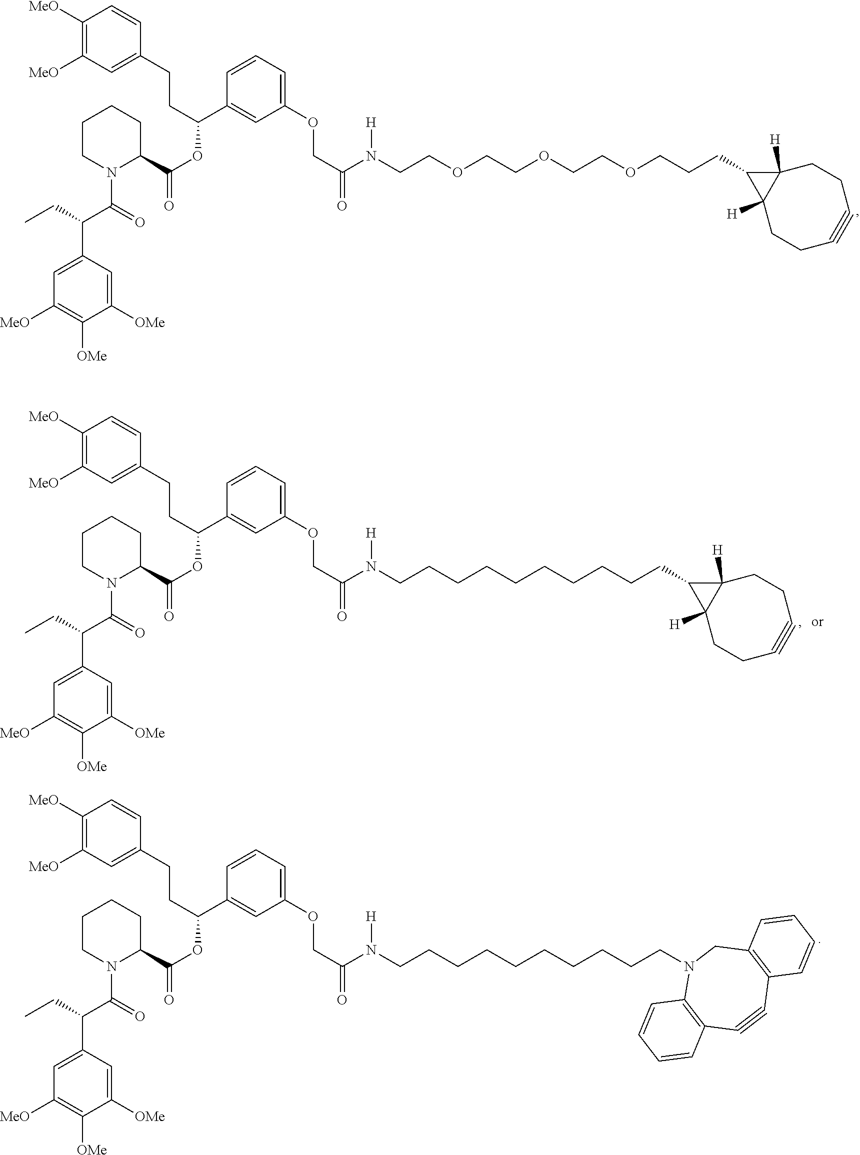

25. A method of labeling a cell having an azide-modified sugar on its surface, the method comprising contacting the cell with a compound having the formula: 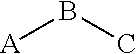
wherein:
A is a small molecule ligand that binds to an FKBP binding site;
B is a chemical linker chosen from an alkyl, an alkenyl, an amide, an ester, a thioester, a ketone, an ether, a thioether, a disulfide, an ethylene glycol unit; a cycloalkyl, a benzyl, a heterocyclic, a maleimidyl, a hydrazone, a urethane, an azole, an imine, a haloalkyl, or a carbamate, or any combination thereof; and
C is an azide reactive molecule chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane.

26. The method according to claim 25, further comprising contacting the cell with the azide-modified sugar prior to contacting the cell with the compound.

27. The method according to claim 25 or claim 26, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

28. The method according to claim 27, wherein the azide-modified sugar is acetylated at 1, 2, 3, or 4 positions.

29. The method according to any one of claims 25 to 28, wherein the FKBP binding site is the FK506-FKBP binding site or the mutant (F36V) FKBP binding site,

30. The method according to claim 29, wherein the FKBP binding site is the FK506-FKBP binding site.

31. The method according to claim 29, wherein the FKBP binding site is the mutant (F36V) FKBP binding site.

32. The method according to any one of claims 25 to 28, wherein the small molecule ligand is

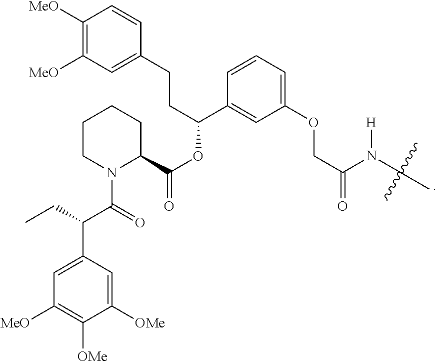 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

33. The method according to any one of claims 25 to 32, wherein the chemical linker is an alkyl or an ethylene glycol unit.

34. The method according to claim 33, Therein the chemical linker is an alkyl.

35. The method according to claim 34, wherein the alkyl is a C₂-C₁₆alkyl.

36. The method according to claim 35, wherein the alkyl is a C₄-C₁₂alkyl or a C₄-C₁₆alkyl.

37. The method according to claim 36, wherein the alkyl is a C₄-C₁₀alkyl.

38. The method according to claim 37, wherein the alkyl is C₄alkyl or C₁₀alkyl.

39. The method according to claim 33, wherein the chemical linker is an ethylene glycol unit.

40. The method according to claim 39, wherein the ethylene glycol unit is a polyethylene glycol (PEG).

41. The method according to claim 40, wherein the PEG is PEG2 to PEG16.

42. The method according to claim 41, wherein the PEG is PEG2, PEG3, or PEG4.

43. The method according to any one of claims 25 to 42, wherein the azide reactive molecule is chosen from a cyclooctyne, a cyclooctene, and a tetrazine.

44. The method according to claim 43, wherein the cyclooctyne is dibenzocyclooctyne (DBCO), bicyclo[6.1.0]nonyne (BCN), monofluorinated cyclooctyne, difluorocyclooctyne, dimethoxyazacyclooctyne, dibenzoazacyclooctyne, biarylazacyclooctynone, 2,3,6,7-tetramethoxy-dibenzocyclooctyne, sulfonylated dibenzocyclooctyne, carboxymethyl monobenzocyclooctyne, or pyrrolocyclooctyne.

45. The method according to claim 43, wherein the cyclooctene is trans-cyclooctene (TCO).

46. The method according to claim 43, wherein the tetrazine is methyltetrazine, diphenyltetrazine, 3,6-di-(2-pyridyl)-s-tetrazine, 3,6-diphenyl-s-tetrazine, 3-(5-aminopyridin-2-yl)-6-(pyridin-2-yl)-s-tetrazine, or N-benzoyl-3-(5-aminopyridin-2-yl)-6-(pyridin-2-yl)-s-tetrazine.

47. The method according to claim 25, wherein:
the FKBP binding site is the FK506-FKBP binding site or the mutant (F36V) FKBP binding site;
the chemical linker is an alkyl or an ethylene glycol unit; and
the azide reactive molecule is chosen from a cyclooctyne, a cyclooctene, and a tetrazine.

48. The method according to claim 25, wherein:
the FKBP binding site is the FK506-FKBP binding site or the mutant (F36V) FKBP binding site;
the chemical linker is a C₂-C₁₆alkyl or a polyethylene glycol which is PEG2 to PEG16; and
the azide reactive molecule is DBCO, BCN, TCO, or methyltetrazine.

49. The method according to claim 25, wherein:
the FKBP binding site is the mutant (F36V) FKBP binding site;
the chemical linker is a C₄-C₁₀alkyl or a polyethylene glycol which is PEG2, PEG3, or PEG4; and
the azide reactive molecule is DBCO, BCN, TCO, or methyltetrazine.

50. The method according to claim 25, wherein:
the small molecule ligand is 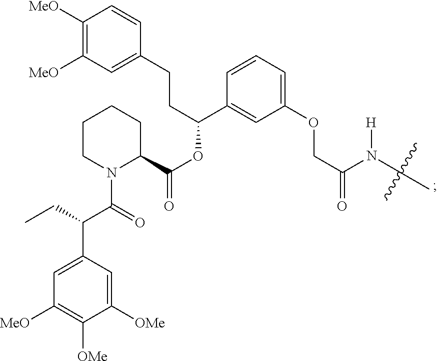 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]
the chemical linker is C₄alkyl, C₁₀alkyl, or PEG3; and
the azide reactive molecule is DBCO or BCN.

51. The method according to claim 25, wherein the compound comprises the formula:

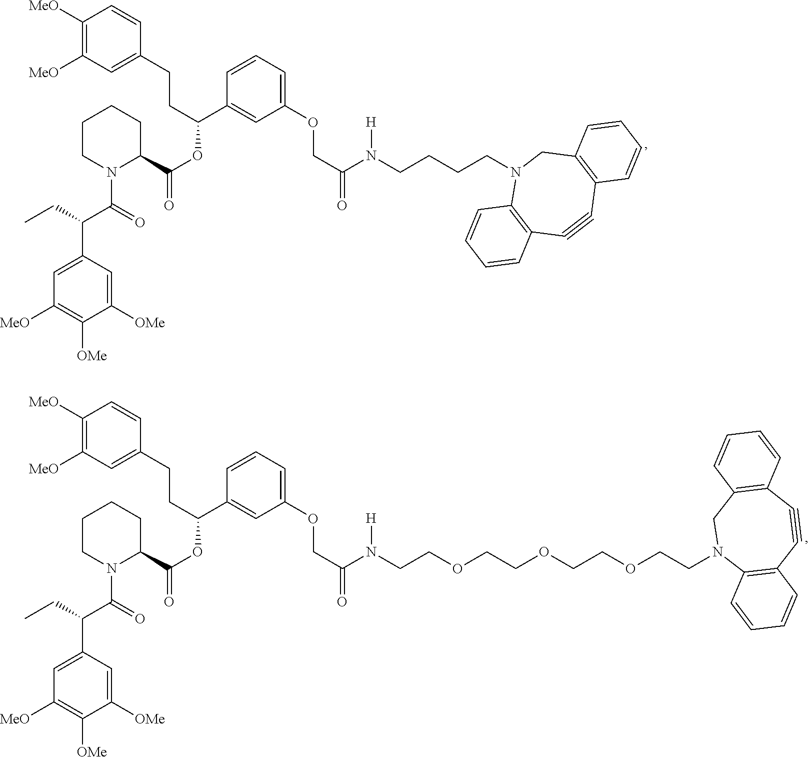 [SMILES: CC[C@H](C(=O)N1CCCC[C@H]1C(=O)O[C@H](CCc1ccc(OC)c(OC)c1)c1cccc(OCC(=O)NCCCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCN2Cc3ccccc3C#Cc3ccccc32)c1)c1cc(OC)c(OC)c(OC)c1]

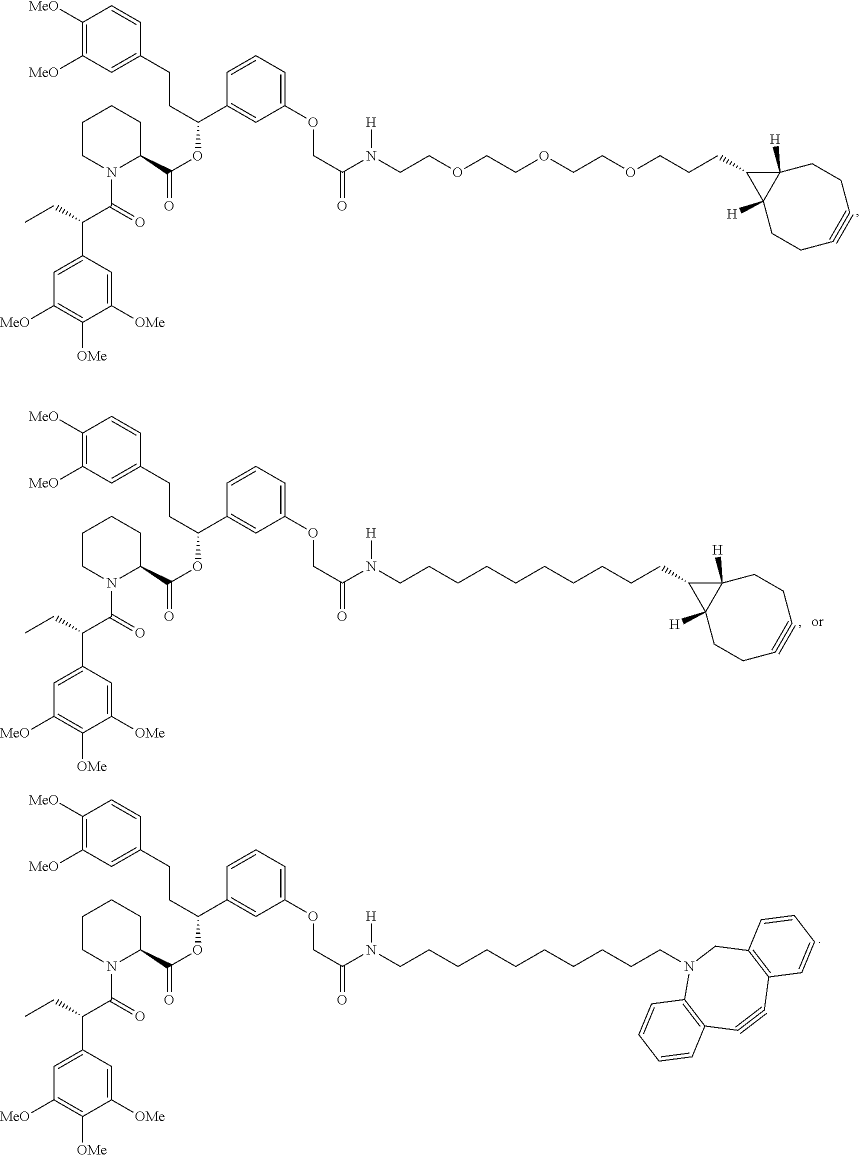

52. The method according to any one of claims 25 to 51, further comprising contacting the cell with a complex, wherein the complex comprises an FKBP binding site linked to a polynucleotide, peptide, or small molecule.

53. The method according to any one of claims 25 to 51, further comprising contacting the cell with a complex, wherein the complex comprises an FKBP binding site linked to a polynucleotide.
